(19) United States
(12) Patent Application Publication
(10) Pub. No.: US 2009/0181150 A1
(43) Pub. Date: Jul. 16, 2009

(54) Cholesterol lowering supplement and low cholesterol egg produced by using the same

(76) Inventors: Jeong hak Kim, Jeonju (KR); Hyeon jin Kim, Jeonju (KR); Seong tshool Hong, Jeonju (KR)

Correspondence Address: COLLARD & ROE, P.C., 1077 NORTHERN BOULEVARD, ROSLYN, NY, 11576, US

(21) Appl. No.: 11/658,929

(22) PCT Filed: Dec. 10, 2004
(86) PCT No.: PCT/KR04/03247

§ 371 Date: Jan. 30, 2007

(30) Foreign Application Priority Data
Jul. 30, 2004 (KR) 10 2004-0060480

(51) Int. Cl. A23L 1/32; A61K 31/366; A61K 31/216; A61K 35/74

(52) U.S. Cl. 426/614; 514/460; 514/510; 424/780

(57) ABSTRACT

This invention provides compositions and methods for producing low cholesterol poultry eggs using hypocholesterolemic compounds, cholesterol lowering supplements, and feeds therefrom. Preferably, this invention provides microbial statins without methyl group in C6, as shown in formula V, in order to produce low cholesterol poultry eggs.

The invention allows producing low cholesterol poultry eggs without concomitant reduction of egg production by providing microbial statins, compactin and pravastatin as well as their derivatives, as cholesterol lowering components. This invention also provides methods for producing low cholesterol poultry eggs without huge increase of production cost since the effective amount of cholesterol lowering composition containing microbial statin costs 1/20-¼ of that of synthetic statin.

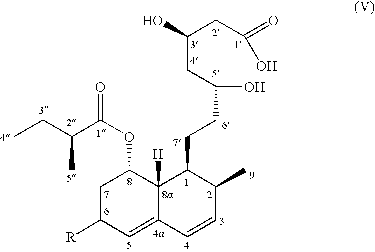

### TECHNICAL FIELD

This invention relates to compositions and methods for producing low cholesterol poultry eggs using hypocholesterolemic compounds, cholesterol lowering supplements, and feeds therefrom.

### BACKGROUND ART

Cholesterol is a kind of lipid in every animal product. It is an essential constituent of cell membranes in human and serves as a starting material for the synthesis of important biological compounds such as steroid hormones and bile acids. However, it is important to maintain the suggested level of serum cholesterol since exceed amount of cholesterol can be harmful.

Specifically, high serum cholesterol levels are termed hypercholesterolemia (more than 200 mg/dL of blood cholesterol), which is a common chronic disease found in 52% of adults in the world. Hypercholesterolemia is a major risk factor for arteriosclerosis, which leads inter alia to myocardial infarction, angina pectoris, hypertension, and stroke. Currently, coronary artery disease is the leading cause of human mortality in the United States and in many other developed countries although it was the forth cause of death in 1900s. It is generally accepted that high levels of cholesterol in the human diet due to more frequent use of animal products such as eggs, meats, and dairy products (milk and butter) can result in a rise in serum cholesterol and thereby increases the risk of cardiovascular diseases.

However, the consumption of cholesterol-rich animal products increases every year and it seems very difficult to limit the intake of animal products significantly. Therefore, research and development efforts have been directed to low cholesterol foods that can provide both basic nutrition and may prevent coronary heart disease.

Chicken eggs are an excellent foodstuff from a nutritional standpoint due to their composition of high-quality protein, saturated fatty acids, mono- and polyunsaturated fatty acids, minerals, and vitamins. However, in addition to these essential dietary components, eggs contain about 200 mg of cholesterol per egg and have been considered a major source of dietary cholesterol. Because of recent understanding of the association between total plasma cholesterol levels and the incidence of coronary heart disease (CHD), it is surmised that an increased amount of dietary cholesterol may increase risk of CHD (Weggemans et al., 2001). Growing numbers of health-conscious consumers exclude eggs from their diets in an effort to limit daily cholesterol consumption to 300 mg/day as recommended by the American Heart Association (National Institutes of Health Consensus Development Panel, 1985). Hence there has been a steady decrease of per capita egg consumption in developed countries. In the US, increasing public concern over dietary cholesterol is reflected in annual per capita egg consumption, which has declined from 303 to 256 during the past 35 years (US Department of Agriculture, 2002). Eggs with reduced cholesterol content may be an attractive, highly nutritious food for health-conscious consumers and a lucrative product for egg producers. This invention provides compositions and methods for producing low cholesterol poultry eggs using hypocholesterolemic compounds, cholesterol lowering feed supplements, and feeds therefrom.

In previous art of U.S. Pat. No. 6,177,121, it was shown that low cholesterol eggs were produced by administration of purified lovastatin, simvastatin, or atorvastatin (Elkin et al., J. Nutr. 1999:129:1010-1019, Elkin et al., J. Agric. Food Chem. 2003:51:3473-3481). Although the cholesterol-lowering effect was found to be satisfactory when the egg-laying chickens are fed 0.03˜0.06% of simvastatin or atorvastatin for 5 weeks, use of these methods is impractical due to the high price of effective amounts of statins, and consequently unmarketable production cost of low cholesterol eggs. In addition, oral administration of simvastatin and atorvastatin decrease egg production, increasing production cost of low cholesterol eggs again.

In the same art, lovastatin was shown to reduce egg cholesterol level ineffectively, showing only 3˜6% reduction at its high dose. Therefore, it is very difficult to commercialize low cholesterol eggs using lovastatin, simvastatin, or atorvastatin as key components of cholesterol lowering feed supplements.

Statins are cholesterol lowering agents by inhibition of the biosynthesis of cholesterol restraining 3-Hydroxy-3-Methylglutaryl coenzyme A reductase (HMG-CoA reductase). Cholesterol is synthesized by multi-step biosynthesis starting from acetyl-CoA in humans and livestock, warm-blooded animals. The key rate-limiting step in cholesterol biosynthesis is to convert 3-hydroxy-3-methylglutaryl-coenzyme A to mevalonic acid by the key enzyme known as 3-hydroxy-3-methylglutaryl-coenzyme A reductase (HMG-CoA reductase, Formula 1).

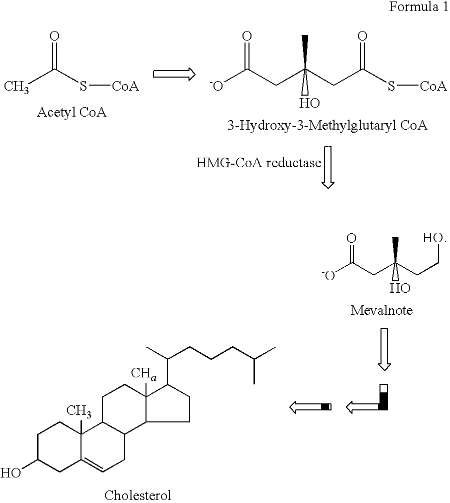

Statin inhibits HMG-CoA reductase due to its structural similarity to mevalonic acid, a substrate of HMG-CoA reductase. Several statins have been developed as cholesterol lowering agents for the treatment of hypercholesterolemia. These include mevastatin (disclosed in U.S. Pat. No. 3,983,140), lovastatin (disclosed in U.S. Pat. No. 4,231,938), pravastatin (disclosed in U.S. Pat. No. 4,346,227), simvastatin (disclosed in U.S. Pat. Nos. 4,444,784 and 4,450,171), fluvastatin (disclosed in U.S. Pat. No. 4,739,073), atorvastatin (disclosed in U.S. Pat. No. 5,273,995), and cerivastatin (disclosed in U.S. Pat. No. 5,502,199).

Among above statins, compactin (mevastatin), lovastatin, and pravastatin are produced by microbial culture while other statins are synthesized. Microbial statins are classified by R group at C[6] as compactin (R:—H), lovastatin (R:—CH3), and pravastatin (R:—OH). Statins can be lactone structure, acid forms (formula V) or salt.

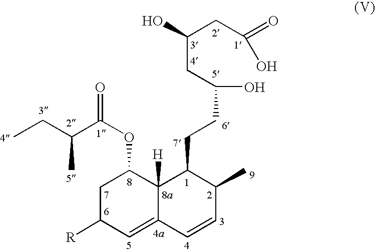

Compactin, having a hydrogen atom in C6 in the formula V, has been isolated in the culture medium of Penicillium (U.S. Pat. No. 3,983,140). Thereafter, several compactin-producing strains were reported in various species, including P. citrinum, P. brevicompactum, P. cyclopium, P. adametzioides, Trichoderma viridae, Aspergillus terreus, Gliocladium sp. (U.S. Pat. Nos. 3,983,140; 4,049,495; 4,137,322; 5,691,173; Korean Pat. No. 832801; 832329; 10-0378640). Unfortunately, the development of compactin as hypocholesterolemic drugs was discontinued after clinical trials showing serious toxicity of compactin. Then, other compounds found to be structurally related to compactin have been isolated and studied for hypocholesterolemic activity. These derivatives are 3-hydroxy compactin, 6-hydroxy compactin, 8a-hydroxy compactin, 4a,5-dihydrocompactic acid, 5′-phosphocompactic acid, and ML-236A (Chakravarti et al., 2004, Appl. Microbial. Biotechnol. 64:618-624).

Pravastatin, having a hydroxyl group in C6 in the formula V, has been shown to have hypocholesterolemic effect. Unlike compactin, however, pravastatin is not toxic and has been developed as prescription drugs for hypercholesterolemia patients. Currently, pharmaceutical pravastatin is produced by enzymatic bioconversion process in which hydrogen group in C6 of compactin is converted to hydroxyl group by microorganisms (U.S. Pat. No. 4,346,227). In previous arts, several species in the genera of Streptomyces, carbophilus, S. roseochromogenus subsp., Nocardia and Actinomadura were reported to convert compactin to pravastatin by bioconversion process (U.S. Pat. Nos. 5,942,423; 5,179,013; 4,537,859; 4,448,979; 4,346,227; Canadian Pat. No. 1,150,170; 1,186,647; Korean Pat. No. 10-0414334; 10-0180706; Serizawa et al., 1983, J. Antibiotics 36: 887-891).

### DISCLOSURE

### Technical Problem

The invention provides economic and effective methods for producing low cholesterol eggs, having lower than naturally-occurring levels of cholesterol.

### Technical Solution

The invention provides cholesterol lowering composition effective in reducing the cholesterol content in poultry eggs with a little amount of supplementation without reduction of egg production. The invention differs with previous arts in which supplementation of atorvastatin or simvastatin caused reduction of egg production which resulted in a rise of production cost. Statins without methyl group in C6 of formula V, however, reduce egg cholesterol at 1/10 to ½ amounts of atorvastatin, simvastatin, and lovastatin for the same effects.

The invention provides poultry cholesterol lowering feed supplements, cholesterol lowering feeds, and produced low cholesterol eggs products therefrom as well as the methods.

The present invention may be better understood with reference to the accompanying examples that are intended for purposes of illustration only and should not be construed to limit the scope of the invention, as defined by the claims appended hereto.

### BEST MODE

As used herein, the following terms have the following meanings:

“Poultry” is intended to mean any domesticated poultry raised for human consumption, including chicken, quail, duck, goose, ostrich and turkey.

“Egg” is intended to mean any egg products for human consumption from domesticated poultry, including chicken, quail, duck, goose, ostrich and turkey.

“Low cholesterol egg products” is intended to encompass egg products with reduced cholesterol content compared to eggs produced by conventional husbandry methods.

This invention provides compositions for reducing the cholesterol content in poultry eggs. In one aspect of the invention, the composition comprises cholesterol lowering components selected from the group consisting of: compactin and its derivatives of the formula I, compactin and its derivatives of the formula II, a pharmaceutically acceptable salt of said compactin and its derivatives, and an ester of said compactin and its derivatives. The compactin is characterized as statins having only hydrogen groups at C6 of formula I or II.

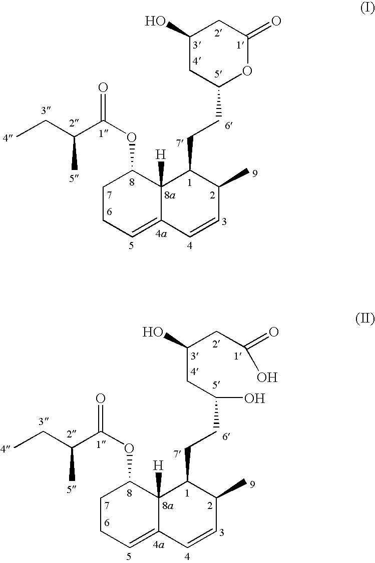

Compactin is called as mevastatin or ML236B and includes lactone structure (formula I), free acid structure (formula II), salt and esters therefrom. The compactin derivatives are any statins with a hydrogen group in C6 of formula V, including 3-hydroxy compactin, 6-hydroxy compactin, 8a-hydroxy compactin, 4a,5-dihydrocompactic acid, 5′-phosphocompactic acid, ML-236A. Compactin derivatives are also microbial inhibitors of cholesterol biosynthesis but are not limited thereto. Compactin producing strains include Streptomyces roseochromogenus for 3-hydroxy compactin, Mucor hiemalis for 6-hydroxy compactin, Schizophyllum commune for 8a-hydroxy compactin, Penicillum citrinum for 4a, 5-dihydrocompactic acid, Carcinella muscae for 5′-phosphocompactic acid, and Emericella unguis for ML-236A.

In another embodiment of the invention, the composition for reducing the cholesterol content in eggs, comprises cholesterol lowering components selected from the group consisting of: pravastatin and its derivatives of the formula III, pravastatin and its derivatives of the formula IV, a pharmaceutically acceptable salt of said pravastatin and its derivatives and an ester of said pravastatin and its derivatives. The pravastatin is characterized as statins having a hydroxyl group at C6 of formula III or IV.

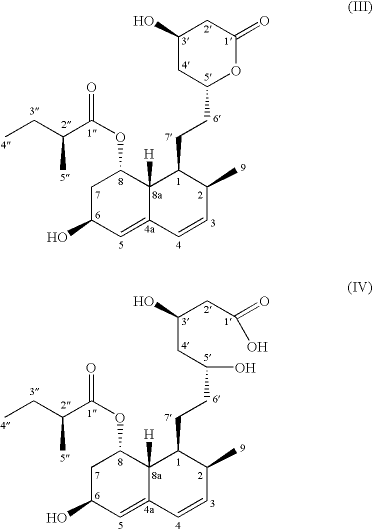

Pravastatin, called as eptastatin, mezalotin, or pravachol, is produced by bioconversion from microorganism. Pravastatin in this art includes lactone structure (formula III), free acid structure (formula IV), salt and esters therefrom. The pravastatin derivatives are any statins with a hydroxyl group in C6 of formula V.

This invention also provides poultry feed supplements and feed containing the said cholesterol lowering compositions.

This invention also provides a method of producing low cholesterol eggs comprising administering to poultry a composition containing cholesterol lowering components selected from the group consisting of: compactin and its derivatives of the formula I, compactin and its derivatives of the formula II, a pharmaceutically acceptable salt of said compactin and its derivatives, and an ester of said compactin and its derivatives.

Preferably, poultry animals are fed cholesterol lowering supplements of the invention, said compactin and its derivatives independently or by mixing with the conventional animal feed for the final amount of 0.0001-3% by weight. A preferred feeding schedule is to feed the animal at least once a day with the supplemented feed for a period of more than 5 days duration although the minimum duration and feeding amount to produce low cholesterol eggs can be adjusted depending on the poultry species.

This invention also provides a method of producing low cholesterol eggs comprising administering to poultry a composition containing cholesterol lowering components selected from the group consisting of: pravastatin and its derivatives of the formula III, pravastatin and its derivatives of the formula IV, a pharmaceutically acceptable salt of said pravastatin and its derivatives, and an ester of said pravastatin and its derivatives.

Preferably, poultry animals are fed cholesterol lowering supplements of the invention, pravastatin and its derivatives of the formula (III), formula (IV), salt and ester form, independently or by mixing with the conventional animal feed for the final amount of 0.0001-3% by weight.

This invention also provides derivatives of compactin and pravastatin which can be any intermediates obtained during hypocholesterolemic microbial culture and subsequent purification process of microbial statins.

Compactin, pravastatin, and their derivatives are not synthetic statins but microbial statins which are products of microbial culture. Thus, manufacturing of compactin and pravastatin for pharmaceutical purpose starts with the culture of microorganisms followed by purification of statins from the culture media, containing several statin-derived intermediates.

Manufacturing of pravastatin, for example, starts with the culture of C-6 hydroxylation microorganisms, such as Streptomyces. Next, compactin was added into the Streptomyces culture to initiate bioconversion of compactin to pravastatin via C-6 hydroxylation. After bioconversion reaction, the culture broth was recovered by centrifugation of culture for column chromatography to yield pure pravastatin. The culture broth for column chromatography usually contains pravastatin as well as unused compactin and other derivatives due to low conversion rate of about 40-70% and incomplete hydroxylation. Streptomyces cell precipitates after removal of broth also contains compactin, pravastatin, and other derivatives in small amounts. In addition, prewashing solution, washing solution and pass-through during column purification also contains compactin, pravastatin, and other derivatives in small amounts due to incomplete recovery of statin.

These statin-containing intermediates from manufacturing process of pharmaceutical drugs can be applied directly to animal although its use for human is limited without further purification. Therefore, side products from manufacturing pharmaceutical statins can be used as cholesterol lowering supplements to produce low cholesterol eggs at economic price. Preferably, these cholesterol lowering supplements can be supplied as solid form after lyophilization.

As used herein, “industrial intermediates or by-products” is intended to encompass any products obtained from industrial microbial culture containing hypocholesterolemic compounds, including every intermediates from each production steps, such as microbial culture, statin-containing broth, microbial cell precipitates, washed solution from preparative column, solution from column elution, etc.

The present invention may be better understood with reference to the accompanying examples that are intended for purposes of illustration only and should not be construed to limit the scope of the invention, as defined by the claims appended hereto.

### Example 1

### Low Cholesterol Eggs by Compactin

(1) Administration of cholesterol lowering supplements

Healthy ISA brown hens, 45-week old, were assigned randomly to each dietary group (control group, 8 birds; experimental group, 6 birds each). Each bird was placed in an individual cage in an environmentally controlled room (25° C., 50% relative humidity, 16L:8D). The hens were allowed for 2 weeks in which to adapt to the feed with no additives and the housing. Control birds were fed a commercial diet based on corn and soybean meal (Table 1), while birds in experimental groups were fed diets supplemented with 0.003 or 0.03% of compactin (Sigma Aldrich Korea) for 6 weeks. Feed and water were provided ad libitum throughout the experiment. Feed consumption, egg production and egg weight were recorded daily. Egg production was expressed as percent hen day production; (100×number of eggs laid)/(number of hens×days).

TABLE 1

| Ingredients | % |
|---|---|
| Crude protein (not less than) | 14.50 |
| Crude fat (not less than) | 2.50 |
| Crude fiber (not less than) | 7.00 |
| Crude ash (not less than) | 14.80 |
| Calcium (not less than) | 3.50 |
| Phosphorus (not less than) | 0.35 |
| Methionine and Cysteine (not less than) | 0.50 |
| ME (kcal/kg) | 2,600 |

### (2) Egg Laying Performance

The effects of 0.003% or 0.03% compactin on the laying performance of 45-week old ISA brown hens were investigated (Table 2). Egg weight was maintained at more than 60 grams after 6 weeks of oral administrations of compactin at both doses. Feeding of 0.003% or 0.03% compactin did not cause any difference in egg production compared to the control group without administration of compactin. Maintenance of laying performance with compactin feeding is of contrast to the previous art in which atorvastatin reduced egg production down to 70% after feeding for 5-week.

TABLE 2

| Compactin added | | |
|---|---|---|
| (weight %) | Egg weight (g) | Egg production (%) |
| 0.000 | 66.0 ± 0.5 | 86 |
| 0.003 | 64.2 ± 0.3 | 84 |
| 0.03 | 61.5 ± 0.5 | 81 |

### (3) Egg Cholesterol Analysis

For egg cholesterol analysis, eggs from each bird were collected and egg yolk was separated, weighed, and sampled for analysis. Lipids from eggs were extracted by the method of Folch et al. (1957). Briefly, one gram of sample was saponified with 3 mL of 33% KOH and incubated with 30 mL of 95% methanol in a 65° C. water bath for 90 min. After saponification, 10 mL hexane and 3 mL water were added, followed by vigorous shaking for 10 min. 5α-cholestan (Sigma C-8300) was used as an internal standard. Cholesterol content was determined by a gas chromatography (Shimadzu, Japan) using column VB-1 (30 m×0.25 mm×0.25 μm, VICI Inc.) with a split ratio of 100:1 and nitrogen as carrier gas for a column flow rate of 0.54 mL/min. Injector, column, and detector temperatures were 275° C., 290° C., and 340° C., respectively. Table 3 shows the effect of compactin on egg cholesterol. The average control yolk weight was 17.2 g. Yolk weight showed a slight decrease with compactin administration compared to the control. Administration of compactin caused the significant reduction in yolk weight, compared to the control with 12.8 mg cholesterol per gram of yolk. When expressed as total yolk cholesterol content, percentage changes compared to that of control were 16% and 29% at 0.003% and 0.03% compactin, respectively. It should be noted that low cholesterol egg can be produced by administration of compactin less than 0.03%, indicating compactin is 2 or 3 times more effective than atorvastatin in producing low cholesterol eggs.

TABLE 3

| Compactin | | Cholesterol | Cholesterol | Cholesterol |
|---|---|---|---|---|
| added | Egg york | concentration | content | content |
| (weight %) | weight (g) | (mg/g) | (mg/egg) | change (%) |
| 0.000 | 17.2 ± 0.4 | 12.8 ± 0.1 | 220 | — |
| 0.003 | 16.5 ± 0.4 | 11.2 ± 0.3 | 185 | −16 |
| 0.03 | 14.9 ± 0.7 | 10.5 ± 0.2 | 156 | −29 |

### Example 2

### Low Cholesterol Eggs by Pravastatin

Egg production and yolk cholesterol analysis was performed with 0.003 and 0.03% pravastatin administered group with the same procedure as described in Example 1 above. Hen performance was investigated by measuring egg weight and egg production as in Table 4. Pravastatin group had maintained above 80% of egg production rate after 6-week administration, same as control. This is in contrast with previous art in which atorvastatin resulted in decrease of egg production rate more than 15% compared to control.

TABLE 4

| Pravastatin added | | |
|---|---|---|
| (weight %) | Egg weight (g) | Egg production (%) |
| 0.000 | 66.2 ± 0.5 | 86 |
| 0.003 | 62.5 ± 0.3 | 84 |
| 0.03 | 58.8 ± 0.7 | 82 |

Table 5 shows the effect of pravastatin on egg cholesterol. Administration of pravastatin caused reduction in both yolk weight and cholesterol concentration compared to control, resulting decrease of total cholesterol content. Administration of 0.03% pravastatin produce eggs with 24% less cholesterol compared to control.

TABLE 5

| Pravastatin | | Cholesterol | Cholesterol | Cholesterol |
|---|---|---|---|---|
| added | Egg york | concentration | content | content |
| (weight %) | weight (%) | (mg/g) | (mg/egg) | change (%) |
| 0.000 | 17.2 ± 0.4 | 12.8 ± 0.1 | 220 | — |
| 0.003 | 16.5 ± 0.4 | 12.5 ± 0.5 | 207 | −6 |
| 0.03 | 16.1 ± 0.4 | 10.4 ± 0.5 | 167 | −24 |

### Example 3

### Low Cholesterol Eggs by Industrial Products Containing Statin

Streptomyces cell precipitates and cell culture broth after compactin bioconversion were used as cholesterol lowering supplements respectively. First, compactin bioconversion was done as following. Single colony of streptomyces carbophilus was inoculated into 100 ml R2YE in 500 ml erlenmyer flask and incubated at 27° C. at 200 rpm for 3 days. After incubation, 10 ml of seed culture was added to 100 ml of conversion media (glucose 2.0%, corn steep liquor 0.2%, K₂HPO₄ 0.15%, yeast extract 0.1%, MgSO₄.7H₂O 0.15%, ZnSO₄7H₂O 0.001%, NH₄NO₃ 0.1%, peptone 0.1%, pH 7.0) in 500 ml flask and incubated at 27° C. at 200 rpm for 3 days. Then, filter sterilized compactin salt was added into culture up to 0.1% weight of culture and incubated for another 4 days. After biovonversion reaction, culture media was analyzed by TLC to confirm the presence of compactin and pravastatin. This culture was freeze dried for 24 hours and then used as cholesterol lowering supplements. Also Streptomyces cells were obtained from bioconversion reaction by centrifugation at 3,000×g. Streptomyces cell precipitates recovered as pellets was dried at 60° C. and used as cholesterol lowering supplements. Streptomyces cell precipitates as well as bioconversion reaction broth was dried and used as cholesterol lowering supplements as shown in Example 1. Egg production and cholesterol amount were measured afterward.

Egg production and yolk cholesterol analysis was performed with the same procedure as described in Example 1 above except 0.5 and 1% of Streptomyces cell precipitates and bioconversion reaction broth were administered instead of compactin. Hen performance was investigated by measuring egg weight and egg production as in Table 6. Both experimental groups showed usual hen performance similar to control group while egg production rate is marginally increased compared to control.

TABLE 6

| Supplement volume | | |
|---|---|---|
| (weight %) | Egg weight (%) | Egg production (%) |
| Control group | 66.2 ± 0.5 | 85 |
| Dried broth material 0.5 | 63.5 ± 0.8 | 85 |
| Dried broth material 1.0 | 65.1 ± 0.3 | 88 |
| Dried cell material 0.5 | 64.4 ± 0.9 | 87 |
| Dried cell material 1.0 | 64.6 ± 0.7 | 89 |

Table 7 shows the data of egg cholesterol analysis. Egg cholesterol amount as well as yolk weight and cholesterol concentration were reduced in correlation with the amounts of supplements compared to the control. Administration of dried broth material resulted in, reduction of egg cholesterol about 17% and 24% in 0.5% and 1% supplement group, respectively. Administration of dried cell material resulted in reduction of egg cholesterol about 13% and 17% in 0.5% and 1% supplement group, respectively.

TABLE 7

| Supplement | | Cholesterol | Cholesterol | Cholesterol |
|---|---|---|---|---|
| volume | Egg york | concentration | content | content |
| (weight %) | weight (%) | (mg/g) | (mg/egg) | change (%) |
| Control group | 16.6 ± 0.3 | 12.5 ± 0.1 | 220 | — |
| Dried broth material 0.5 | 15.6 ± 0.5 | 11.8 ± 0.5 | 184 | −17 |
| Dried broth material 1.0 | 15.3 ± 0.2 | 10.9 ± 0.7 | 167 | −24 |
| Dried cell material 0.5 | 17.0 ± 0.6 | 11.3 ± 0.4 | 192 | −13 |
| Dried cell material 1.0 | 16.0 ± 0.4 | 11.5 ± 0.9 | 184 | −17 |

### INDUSTRIAL APPLICABILITY

This invention provides a method for producing low cholesterol poultry eggs without concomitant reduction of egg production. This invention allows minimal increase of production costs, as low as 1/20-¼ of that of synthetic statins by providing compactin, pravastatin and their derivatives. The invention also allows producing low cholesterol poultry eggs by providing industrial products from microbial culture of statin-producing microorganisms as cholesterol lowering supplements. This invention allows providing low cholesterol poultry eggs with economic price, contributing prevention of hypercholesterolemia by decreasing consumption of dietary cholesterol.

1: A composition for reducing the cholesterol content in poultry eggs, comprising cholesterol lowering components selected from the group consisting of:
a. compactin and its derivatives of the formula I
b. compactin and its derivatives of the formula II
c. a pharmaceutically acceptable salt of said compactin and its derivatives
d. an ester of said compactin and its derivatives

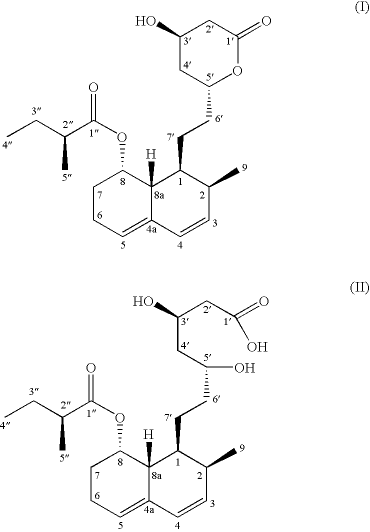

2: The composition of claim 1,
wherein the cholesterol lowering components are any by-products, intermediates, and/or isolates from the industrial preparation of microbial cultures, comprising compactin and its derivatives of formula I or II.

3: A composition for reducing the cholesterol content in poultry eggs, comprising cholesterol lowering components selected from the group consisting of:
a. pravastatin and its derivatives of the formula III
b. pravastatin and its derivatives of the formula IV
c. a pharmaceutically acceptable salt of said pravastatin and its derivatives
d. an ester of said pravastatin and its derivatives

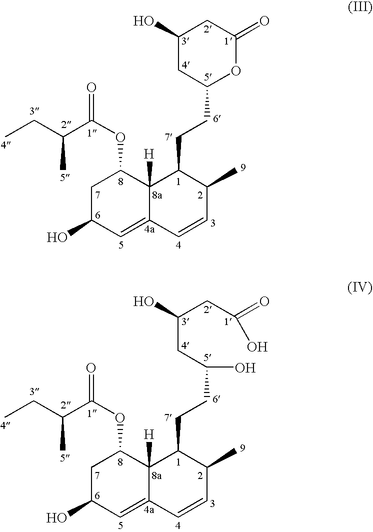

4: The composition of claim 1,
wherein the cholesterol lowering components are any by-products, intermediates, and/or isolates from the industrial preparation of microbial cultures, comprising pravastatin and its derivatives of formula III or IV.

5: Poultry feed supplements, comprising the composition of claim 1.

6: A poultry feed, comprising an oviparous vertebrate animal food and the cholesterol lowering composition of claim 1.

7: Poultry feed supplements, comprising the composition of claim 3.

8: A poultry feed, comprising an oviparous vertebrate animal food and the cholesterol lowering composition of claim 3.

9: A method of producing low cholesterol eggs comprising administering to poultry a composition containing cholesterol lowering components selected from the group consisting of:
a. compactin and its derivatives of the formula I
b. compactin and its derivatives of the formula II
c. a pharmaceutically acceptable salt of said compactin and its derivatives
d. an ester of said compactin and its derivatives

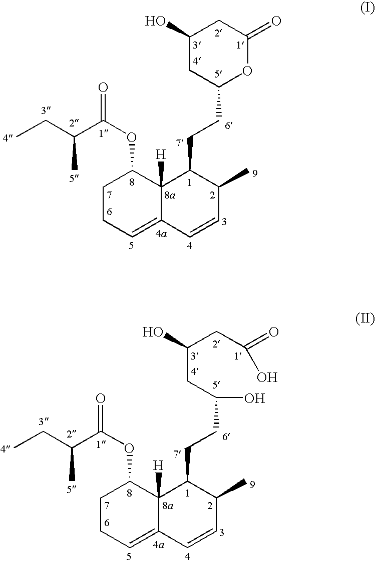

10: The method of claim 9,
wherein the cholesterol lowering components are by-products, intermediates, and/or isolates from the industrial preparation of microbial cultures, comprising compactin and its derivatives of formula I or II.

11: The method of claim 9, wherein poultry feed is supplemented with from 0.0001-3% by weight of the compactin and its derivatives and the poultry is fed the feed at least once per day for more than 5 days.

12: Low cholesterol eggs produced in accordance with the method of claim 9.

13: A method of producing low cholesterol eggs comprising administering to poultry a composition containing cholesterol lowering components selected from the group consisting of:
a. pravastatin and its derivatives of the formula III
b. pravastatin and its derivatives of the formula IV
c. a pharmaceutically acceptable salt of said pravastatin and its derivatives
d. an ester of said pravastatin and its derivatives

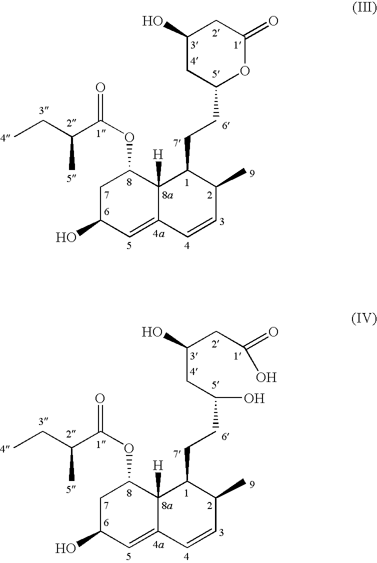

14: The method of claim 13,
wherein the cholesterol lowering components are by-products, intermediates, and/or isolates from the industrial preparation of microbial cultures, comprising pravastatin and its derivatives of formula III or IV.

15: The method of claim 13, wherein poultry feed is supplemented with from 0.0001-3% by weight of the pravastatin and its derivatives and the poultry is fed the feed at least once per day for more than 5 days.

16: Low cholesterol eggs produced in accordance with the method of claim 13.
